(19) United States
(12) Patent Application Publication
(10) Pub. No.: US 2008/0172120 A1
(43) Pub. Date: Jul. 17, 2008

(54) ENDOPROSTHESIS DELIVERY SYSTEMS AND RELATED METHODS

(76) Inventors: Calvin Fenn, Monticello, MN (US); John Chen, Maple Grove, MN (US); Richard J. Olson, Blaine, MN (US)

Correspondence Address: FISH & RICHARDSON PC, P.O. BOX 1022, MINNEAPOLIS, MN, 55440-1022, US

(21) Appl. No.: 11/622,762

(22) Filed: Jan. 12, 2007

(51) Int. Cl. A61F 2/84

(52) U.S. Cl. 623/1.12

(57) ABSTRACT

This disclosure relates to endoprosthesis delivery systems and related methods.

### TECHNICAL FIELD

This disclosure relates to endoprosthesis delivery systems and related methods.

### BACKGROUND

Devices are known for delivering implantable endoprostheses, such as stents, into a body vessel. Devices of this kind often include a proximal portion that remains external to the body vessel during use and a distal portion that, is inserted into the body vessel (e.g., through an incision). The proximal portion typically provides for manipulation of the device during use. The distal portion often includes an outer member slidably positioned about an inner member. With an endoprosthesis disposed therebetween. Generally, the distal portion of the device is advanced through the body vessel to a treatment site (e.g., a stenosis or aneurysm). The outer member can then be retracted to allow the endoprosthesis to expand to engage a wall of the body vessel at the treatment, site. Thereafter, the device is removed leaving the endoprosthesis engaged with the body vessel.

### SUMMARY

In general, this disclosure relates to endoprosthesis delivery systems and related methods. The systems can be used, for example, to deliver an implantable medical endoprosthesis (e.g., a stent) to a treatment site within a body vessel (e.g., a blood vessel) and to deploy the endoprosthesis at the treatment site within the body vessel. The systems can be configured to limit axial movement of the endoprosthesis during deployment of the endoprosthesis. The systems can alternatively or additionally be configured to permit aspiration of fluid (e.g. blood) from the treatment site and/or to permit delivery of one or more therapeutic agents to the treatment site.

In one aspect of the invention, a system includes an inner member and an outer sheath at least partially surrounding the inner member. The inner member and the outer sheath are configured so that an implantable endoprosthesis can be disposed therebetween. The system also includes an expandable member secured to the inner member. The expandable member is configured to inhibit fluid flow through a portion of a body vessel when the expandable member is expanded within the body vessel. The system further includes a lumen in fluid communication with a region of the body vessel adjacent the expandable member during use, wherein fluid can be withdrawn from or injected toward the body vessel through the lumen during use.

In another aspect of the invention, a system includes an inner tubular member and an outer sheath at least partially surrounding the inner tubular member. A self-expanding implantable endoprosthesis is disposed between the inner tubular member and the outer sheath. The system also includes an expandable member secured to the inner tubular member. A proximal face of the expandable member is disposed distal to a distal end of the self-expanding implantable endoprosthesis. The expandable member is configured, when expanded, to substantially prevent distal movement of the self-expanding implantable endoprosthesis during deployment of the self-expanding implantable endoprosthesis.

In a further aspect of the invention, a method includes expanding an expandable member within a body vessel, the expanded expandable member inhibiting fluid flow through a portion of the body vessel. After expanding the expandable member, an implantable endoprosthesis is deployed within the body vessel adjacent the expanded expandable member, fluid is withdrawn from a region of the body vessel adjacent the expanded expandable member.

Embodiments can include one or more of the following features.

In some embodiments, the lumen is defined by the outer sheath.

In certain embodiments, a distal end of the outer sheath includes an opening that fluidly connects the lumen to the region of the body vessel adjacent the expandable member during use.

In some embodiments, the lumen is defined by the inner member.

In some embodiments, a distal end region of the inner member includes an opening that fluidly connects the lumen to the region of the body vessel adjacent the expandable member during use.

The lumen can be placed in fluid communication with the region of the body vessel adjacent the expandable member by proximally displacing the outer sheath, relative to the inner member.

In certain embodiments, the lumen is configured to allow particles dislodged from a wall of the body vessel while deploying an implantable endoprosthesis within the body vessel to pass through the lumen.

In some embodiments, the lumen has a cross-sectional area of about 0.1 mm2 to about 4 mm2.

In some embodiments, the system further includes a suction device in communication with the lumen, the suction device being capable of drawing fluid from the region of the body vessel, adjacent the expandable member via the lumen.

In certain embodiments, the expandable member is configured to substantially prevent fluid flow through the body vessel when expanded therein.

In some embodiments, the expandable member is configured to substantially prevent distal movement of an implantable endoprosthesis disposed between the inner member and the outer sheath during deployment of the implantable endoprosthesis.

In certain embodiments, the inner member includes a bumper member disposed thereon, the bumper member being disposed proximal to the implantable endoprosthesis and being configured to substantially prevent proximal movement of the implantable endoprosthesis during deployment of the implantable endoprosthesis.

In certain embodiments, the implantable endoprosthesis can be deployed by proximally displacing the outer sheath relative to the inner member.

In some embodiments, the implantable endoprosthesis includes a self-expanding stent.

In certain embodiments, the expandable member includes a balloon.

In some embodiments, the expandable member, when expanded, has a diameter that is at least about 75 percent of a diameter of the self-expanding implantable endoprosthesis when fully deployed.

In certain embodiments, the expandable member, when expanded, has a diameter that is about 75 percent to about 100 percent of the diameter of the self-expanding implantable endoprosthesis when fully deployed.

In some embodiments, the proximal face of the expandable member can extends at an angle of about 45 degrees to about 90 degrees relative to a longitudinal axis of the inner member when the expandable member is expanded.

In certain embodiments, the proximal face of the expandable member is positioned within about 5 millimeters of the distal end of the self-expanding implantable endoprosthesis.

In some embodiments, the expandable member is configured to inhibit fluid flow through a region of a body vessel when expanded therein.

In certain embodiments, the lumen is sized to allow particles dislodged from the body vessel while deploying the self-expanding implantable endoprosthesis within the body vessel to pass through, the lumen.

In some embodiments, the expanded expandable member substantially prevents fluid flow through the body vessel.

In certain embodiments, withdrawing the fluid from the body vessel includes activating a suction device in communication with a lumen in fluid communication with the body vessel.

In some embodiments, the fluid is withdrawn from the body vessel after deploying the implantable endoprosthesis.

In certain embodiments, the fluid comprises particles dislodged from the body vessel during deployment of the implantable endoprosthesis.

In some embodiments, deploying the implantable endoprosthesis includes retracting an outer sheath relative to an inner member, the implantable endoprosthesis, prior to deployment, being disposed between the inner member and the outer sheath.

In certain embodiments, the expandable member is secured to the inner member.

In some embodiments, a proximal face of the expandable member is positioned distal to a distal, end of the implantable endoprosthesis.

In certain embodiments, the expanded expandable member can be configured to substantially prevent distal movement, of the implantable endoprosthesis during deployment of the endoprosthesis.

In some embodiments, the method includes delivering a therapeutic agent through the lumen to the region of the body vessel adjacent the expanded expandable member.

Embodiments can include one or more of the following advantages.

In some embodiments, the systems and methods can provide for the prevention of fluid flow through a treatment site within a body vessel during deployment of an endoprosthesis at the treatment site. As a result, downstream flow of fluid and debris (e.g., particles dislodged from the body vessel wall during deployment of the endoprosthesis) can be reduced or prevented. Similarly, the downstream flow of one or more therapeutic agents delivered to the treatment site can be reduced or prevented.

In certain embodiments, the systems and methods can provide for aspiration of fluid from the treatment site following deployment of the endoprosthesis. The aspiration can allow particles dislodged from the body vessel wall during deployment to be collected and removed from the blood stream.

In some embodiments, the systems and method can provide for delivery of one or more therapeutic agents (e.g., anticoagulants, antibiotics, etc.) to the treatment site following deployment of the endoprosthesis. The one or more therapeutic agents can, for example, be used to treat the region of the body vessel in which the implantable endoprosthesis was deployed.

In certain embodiments, the systems and methods can provide for deployment of an implantable endoprosthesis in a body vessel while inhibiting axial movement (e.g., proximal and/or distal movement) of the endoprosthesis during deployment. As a result, the endoprosthesis can be implanted within the body vessel with increased accuracy.

In some embodiments, the systems and methods can provide for delivery and deployment of an implantable endoprosthesis at a treatment site (e.g., an occlusion site) and contemporaneous aspiration of the site and/or delivery of one or more therapeutic agents to the site without removal of the endoprosthesis delivery system. Thus, the overall efficiency of the procedure can be increased.

Other aspects, features, and advantages are in the description, drawings, and claims.

### DESCRIPTION OF DRAWINGS

FIG. 1 is an orthogonal view of an embodiment of a stent delivery system.

FIG. 2 is a plan view of the stent delivery system, of FIG. 1.

FIG. 3 is a detailed cross-sectional view of a distal end region of the stent delivery system of FIG. 1.

FIG. 4 is a plan view of a stent disposed within the distal end region of the stent delivery system of FIG. 1.

FIGS. 5A-5F illustrate a method of using the stent delivery system of FIG. 1 to deliver and implant an implantable stent within a blood vessel.

FIG. 6 is an orthogonal view of an embodiment of a stent delivery system.

FIG. 7 is a detailed partial cross-section of the stent delivery system of FIG. 6 showing an implantable stent disposed within the distal end region of the stent delivery system.

FIG. 8 is a plan view of the stent delivery system of FIG. 6.

FIG. 9 is a detailed cross-sectional view of a distal, end region of the stent delivery system of FIG. 6.

### DETAILED DESCRIPTION

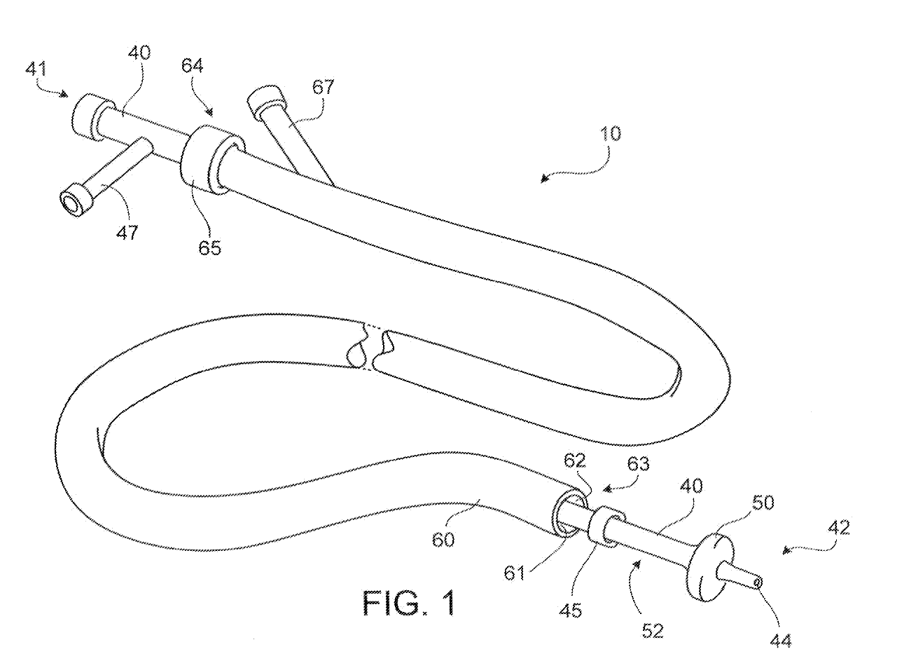

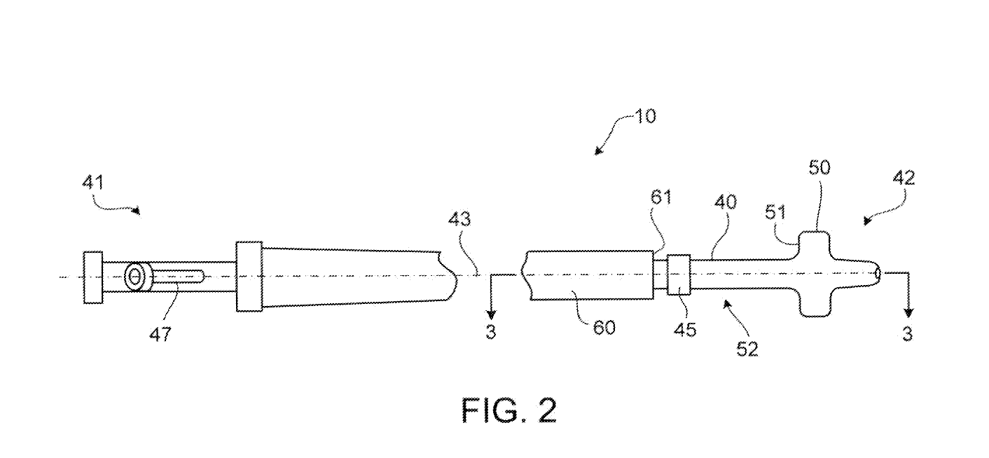

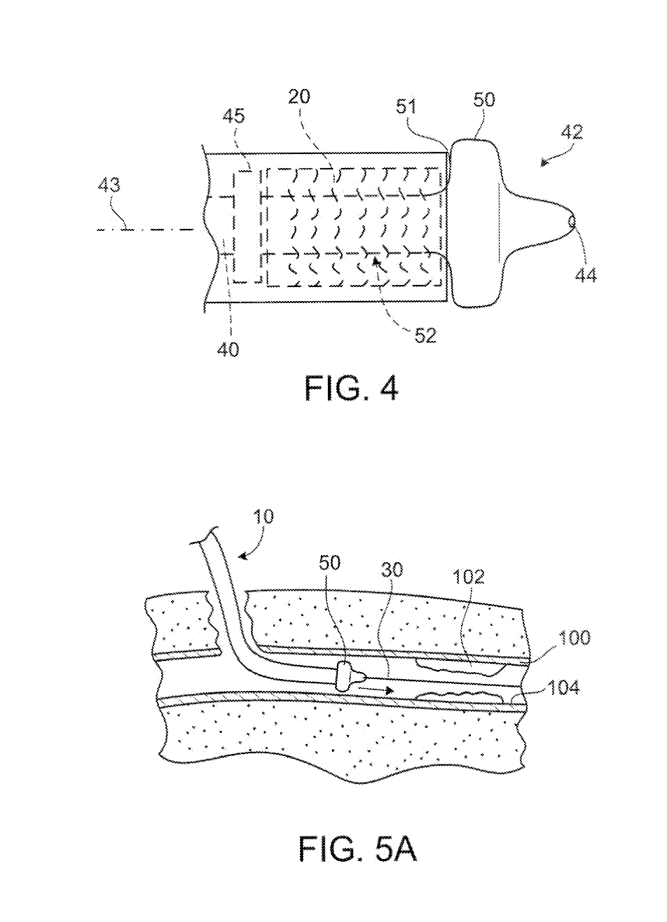

Referring to FIGS. 1 and 2, a stent delivery system 10 includes an inner member 40, a balloon 50 secured to the inner member 40, and an outer sheath 60 surrounding the inner member 40. Outer sheath 60 is partially retracted in FIGS. 1 and 2 to expose a stent bed region 52 of the inner member 40. A self-expanding stent 20, as shown in FIG. 4, can be positioned between the outer sheath 60 and the stent bed region 52 of the inner member 40 during use.

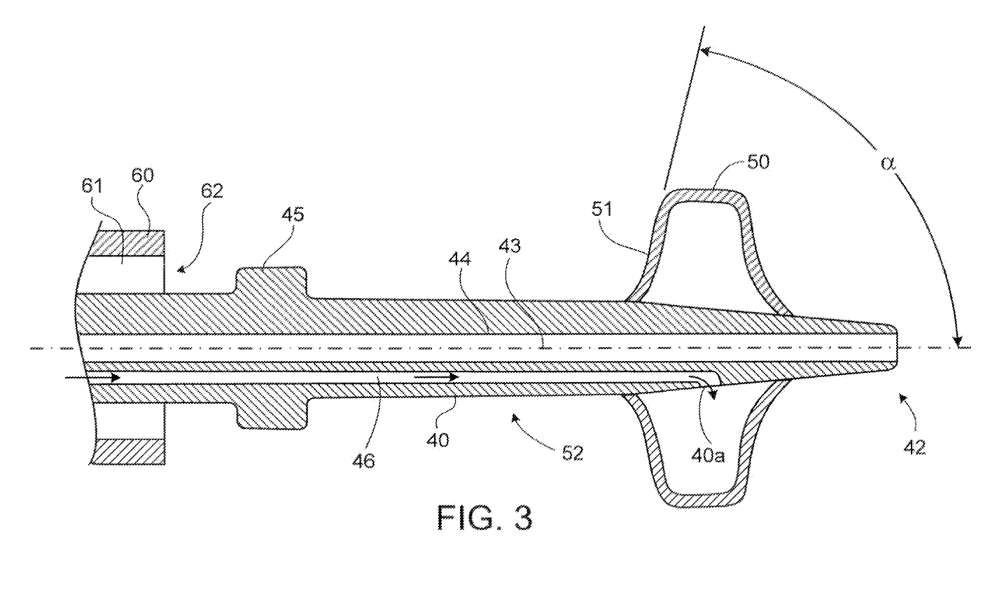

Referring to FIGS. 1-3, the inner member 40 has a proximal end 41, a distal end 42, and a longitudinal axis 43 extending therebetween. As shown in FIG. 3, the balloon 50 is secured to the inner member 40 near the distal end 42 of the inner member 40. The balloon 50 is in fluid communication with an inflation lumen 46 that extends from an opening 40a formed in the side wall of the inner member 40 toward the proximal end 41 of the inner member 40. The opening 40a leads from the inflation lumen 46 to the interior of the balloon 50. A adaptor 47 (shown in FIG. 1) is secured to the proximal end region of the inner member 40 and is in fluid communication with inflation lumen 46. During use, a fluid injection mechanism, such as a syringe, can be connected to the adaptor 47 and used to inject a stream of fluid (e.g., saline and/or radiopaque contrast fluid) into the balloon 50 via the inflation lumen 46 in order to inflate the balloon 50.

Referring to FIG. 3, the balloon 50 is located slightly distal to the stent bed region 52 of the inner member 40. The balloon 50 can be configured so that, when inflated, it inhibits or prevents distal movement of the stent 20 (e.g., distal movement resulting from stmt jumping) during deployment of the stent 20. For example, the balloon 50, when inflated, can have an outer diameter of at least about 75 percent (e.g., about 75 percent to about 110 percent, about 100 percent) of an outer diameter of the stent 20 when fully deployed. The balloon 50 has a proximal face 51 that is positioned distal to a distal end 21 of the stent 20 when the stent 20 is disposed around the stent bed region 52 of the inner member 40. The proximal face 51 can, for example, be positioned no greater than about 5 millimeters (e.g., about 0.01 millimeters to about 5 millimeters) from the distal end 21 of the stent 20 when the stent 20 is disposed around the stent bed region 52 of the inner member 40.

The balloon 50, in addition to being sized to inhibit or prevent distal movement of the stent 20 during deployment, can be shaped to inhibit or prevent distal movement of the stent 20 during deployment. FIG. 3, for example, the proximal face 51 of the balloon 50 can extend at an angle α from about 45 degrees to about 90 degrees (e.g., about 90 degrees) relative to the longitudinal axis 43 of the inner member 40. The relatively steep angle at which the proximal face 51 of the inflated balloon 50 extends can, for example, prevent the stent 20 from, moving beyond the proximal end of the balloon 50 during deployment.

The balloon 50 can alternatively or additionally be configured to substantially inhibit or prevent blood flow within a blood vessel of a patient when the balloon 50 is positioned within the blood vessel and inflated. The balloon 50 can, for example, have an outer diameter, when inflated, that is about 75 percent to about 120 percent (e.g., about 100 percent) of an inner diameter of the blood vessel.

The balloon 50 can include one or more biocompatible polymers suitable for use in a medical device, such as, thermoplastics and thermosets. Examples of thermoplastics include polyolefins, polyamides, such as nylon 12, nylon 11, nylon 6/12, nylon 6, and nylon 66, polyesters, polyethers, polyurethanes, polyureas, polyvinyls, polyacrylics, fluoropolymers, copolymers and block copolymers thereof, such as block copolymers of polyether and polyamide, e.g., Pebax®; and mixtures thereof. Examples of thermosets include elastomers such as EPDM, epichlorohydrin, nitrile butadiene elastomers, silicones, etc. Thermosets, such as expoxies and isocyanates, can also be used. Biocompatible thermosets may also be used, and these include, for example, biodegradable polycaprolactone, poly(dimethylsiloxane) containing polyurethanes and ureas, and polysiloxanes. In some embodiments, the balloon can be formed to have relatively high compliance, such as by controlling material thicknesses, which can provide versatility for use in a broad range of vessel diameters. Other suitable balloon materials are disclosed in U.S. Ser. No. 10/645,014, entitled “Multilayer Medical Device” and filed on Aug. 21, 2003, which is incorporated herein by reference.

In some embodiments, the balloon 50 (e.g., the proximal face 51 of tire balloon 50) includes one or more tacky materials. The proximal face 51 of the balloon 50 can, for example, be formed of one or more tacky materials. Alternatively or additionally, a coating of tacky material can be applied to the proximal face 51 of the balloon 50. The tacky material can increase resistance between the stent 20 and the proximal face 51 of the balloon 50, and thus help to prevent the stent 20 from moving distally during deployment. Examples of tacky materials include urethanes, poly(styrene-b-isobutylene-b-styrene) (SIBS), and elastomers (e.g., Crayton, silicone, etc.). In some embodiments, the balloon can include a polymeric material, and additives can be added to the polymeric material to provide a tacky material.

The balloon 50 can be secured to the inner member 40 using any of various suitable techniques. For example, the balloon 50 can be secured to the inner member 40 using one or more adhesives, such as urethane. Alternatively or additionally, the balloon 50 can be thermally bonded (e.g., welded) to the inner member 40. In some embodiments, the balloon 50 is integrally formed with the inner member 40.

Still Referring to FIG. 3, the inner member 40 forms a guide wire lumen 44, that extends from, the distal end 42 of the inner member 40 toward the proximal end 41 of the inner member 40 along the longitudinal axis 43. A bumper member 45 is secured, to the inner member 40 proximal to the stent bed region 52. The bumper member 45 is configured to substantially prevent proximal movement of the stent 20 during deployment of the stem 20. For example, the bumper member 45 can have an outer diameter that is greater than an inner diameter of the stent 20 in a compressed configuration.

The bumper member 45 can be a tabular member that is disposed around and secured to the inner member 40. Any of various techniques, such as adhesive, thermal bonding, etc., can be used to secure the bumper member 45 to the inner member 40. Alternatively or additionally, the bumper member 45 can be integrally formed with the inner member.

In some embodiments, the bumper member 45 is formed of a polymeric material, which may be relatively incompressible. Exemplary materials include Nylon 12 (e.g., VESTAMID®), a polyether-block co-polyamide polymer (e.g. PEBAX®) or a thermoplastic polyurethane elastomer (e.g., Pellethane™). In certain embodiments, the bumper member 45 is made of a metal or an alloy, such as, stainless steel, Nitinol and/or platinum. The bumper member 45 can be radiopaque and/or can include one or more radiopaque markers.

Referring again to FIG. 1, the stent delivery system 10 includes an outer sheath 60 surrounding and arranged for slidable movement relative to the inner member 40. An aspiration lumen 61 is formed between the outer sheath 60 and the inner member 40. The aspiration lumen 61 extends from an opening 62 at a distal end 63 of the outer sheath 60 toward a proximal end 64 of the outer sheath 60. The outer sheath 60 can have an inner diameter of about 0.5 millimeters to about 3 millimeters, and the inner member 40 can have an outer diameter of about 0.4 millimeters to about 1.5 millimeters. Thus, particles having a diameter of about 0.1 millimeters to about 2.5 millimeters can pass through the annular inflation lumen 61, between the inner member 40 and the outer sheath 60.

An aspiration adaptor 67 is secured to the outer sheath 60 and is in fluid communication with the aspiration lumen 61. During use, a suction device (not shown) can be fluidly connected the aspiration adaptor 67 and activated in order to draw blood out of a blood vessel. For example, as discussed below, during deployment of the stent 20, debris (e.g., plaque particles) may be dislodged from the wail of the blood vessel at the treatment site. The suction device can be configured to draw blood containing the debris from the region of the blood vessel proximal to the balloon 50 via the aspiration lumen 61 to prevent the debris from traveling downstream through the blood vessel of the patient. Examples of suitable suction devices include syringes, vacuum pumps, etc.

A sealing device 65 is located at the proximal end of the outer sheath 60 and can include a gasket between an inner surface 68 of the outer sheath 60 and an outer surface 48 of the inner member 40. The sealing device 65 can provide improved suction at the distal end 63 of the outer sheath 60 and can prevent leakage of blood from the proximal end of the outer sheath 60 during use.

The outer sheath 60 and the inner member 49 can be flexible along their lengths to allow the stent delivery system 10 to be deflected and articulated, e.g., to traverse a tortuous blood vessel. The inner member 40 and outer sheath 60 can include one or more compliant, polymeric materials. Examples of suitable polymeric materials include polyether-block co-polyamide polymers (e.g., PEBAX®), copolyester elastomers (e.g., Amitel® copolyester elastomers), thermoplastic polyester elastomers (e.g., Hytrel.®), thermoplastic polyurethane elastomers (e.g., Pellethane™), polyeolefins (e.g., Marlex® polyethylene, Marlex® polypropylene), high-density polyethylene (HDPE), low-density polyethylene (LDPE), polyamides (e.g., Vestamid® and combinations of these materials. In some embodiments, outer sheath 60 and/or inner member 40 include one or more silicones, in certain embodiments (e.g., when it is desirable to reduce the force used to retract outer sheath 60), outer sheath 60 and/or inner member 40 can be made of a material having a relatively low coefficient of friction (e.g., a fluoropolymer or a silicone). Examples of fluoropolymers include PIPE and FTP. Alternatively or additionally, outer sheath 60 and/or inner member 40 can be made of a material that includes a lubricious additive (e.g., a fluoropolymer, a silicone, an ultrahigh molecular weight polyethylene, an oil or blends thereof).

The stent 20, as noted above, can be a self-expanding stent. Examples of materials from which endoprosthesis 20 can be made include shape memory materials, such as Nitinol, silver-cadmium (Ag—Cd), gold-cadmium (Au—Cd), gold-copper-zinc (Au—Cu—Zn), copper-aluminum-nickel (Cu—Al—Ni), copper-gold-zinc (Cu—Au—Zn), copper-zinc/(Cu—Zn), copper-zinc-aluminum (Cu—Zn—Al), copper-zinc-tin (Cu—Zn—Sn), copper-zinc-xenon (Cu—Zn—Xe), iron beryllium (Fe.sub.3Be), iron platinum (Fe.sub.3Pt), indium-thallium (In—Tl), iron-manganese (Fe—Mn), nickel-titanium-vanadium (Ni—Ti—V), iron-nickel-titanium-cobalt (Fe—Ni—Ti—Co) and copper-tin (Cu—Sn). For yet additional shape memory alloys, see, for example, Schetsky, L. McDonald, “Shape Memory Alloys”, Encyclopedia of Chemical Technology (3rd ed), John Wiley & Sons, 1982, vol. 20, pp. 726-736.

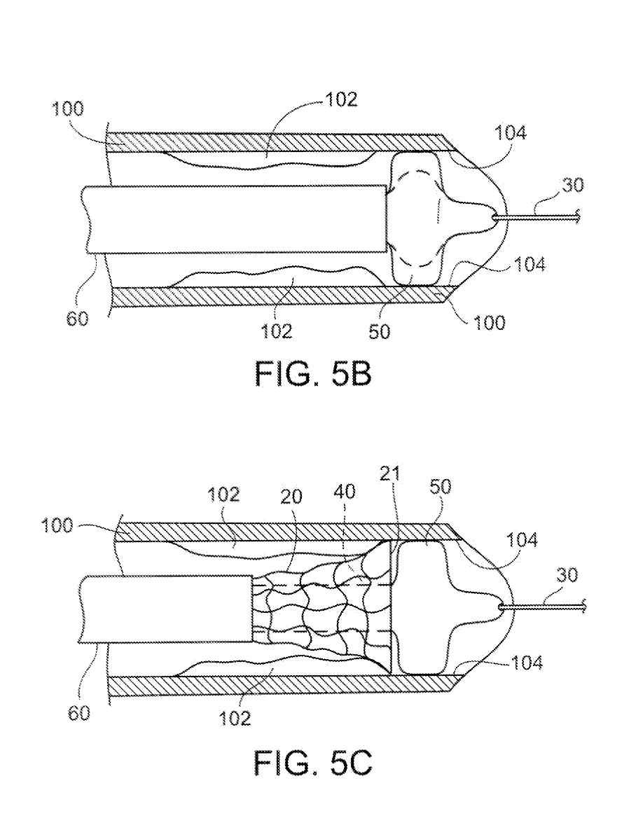

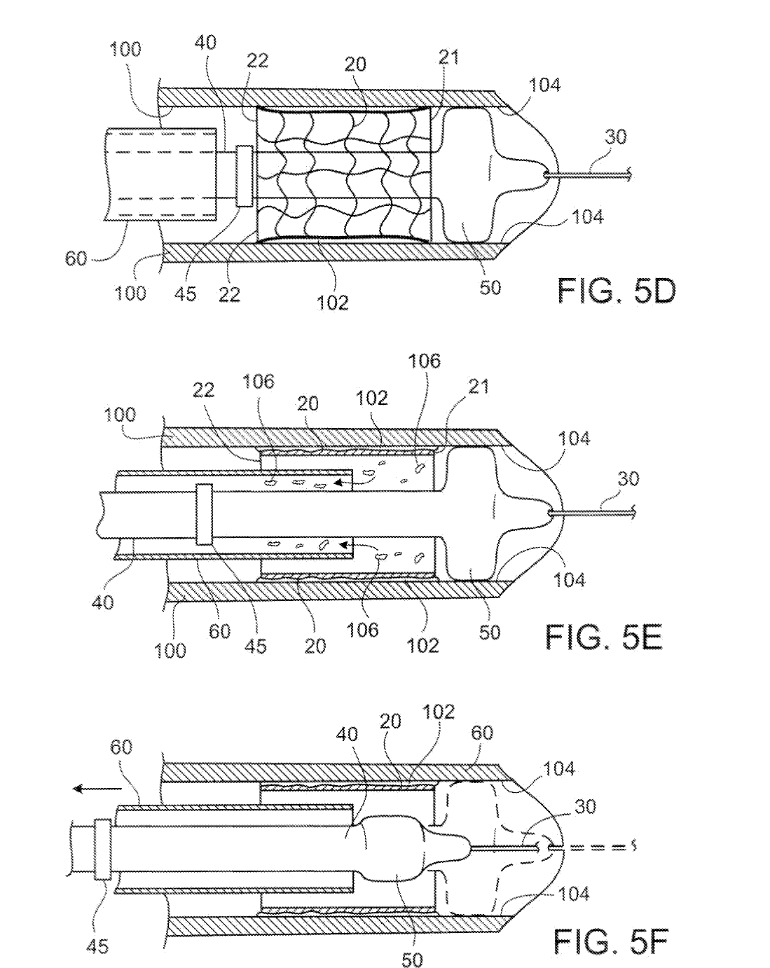

FIGS. 5A-5F diagrammatically show a method of using the stent delivery system 10 to implant the self-expanding stent 20 within a blood vessel. As shown in FIG. 5A, a guide wire 30 is first delivered into a blood vessel 100 of a patient, and then the stent delivery system 10, with the stent 20 secured between the inner member 40 and the outer sheath 60, is inserted percutaneously into the blood vessel 100. The stent delivery system 10 is then advanced along the guide wire 30 toward a treatment site (e.g., an occluded region of the blood vessel) 102. The stent delivery system 10 can be guided through the blood vessel 100 by feel or using an image guidance technique, such as fluoroscopy. While the stent delivery system 10 is being advanced through the blood vessel 100, the balloon 50, as shown in FIG. 5A, can be partially inflated to fit against the distal, end 63 of the outer sheath 60. The balloon 50 can, for example, be inflated to an extent such that the outer diameter of the balloon 50 is greater than or equal to the outer diameter of the outer sheath 60. In this partially inflated state, the rounded edges of the balloon 50 can provide for enhanced trackability when navigating the stent delivery system 10 through a tortuous blood vessel. The rounded edges of the partially inflated balloon 50 can, for example, cover the relatively flat and more rigid distal end of the outer sheath 60, helping to prevent contact between the distal end of the outer sheath 60 and the blood vessel wall 104, in some embodiments, the balloon 50 can provide a smooth surface that can engage the wall 104 of the blood vessel while presenting relatively little resistance to forward movement.

The stent delivery system 10 is advanced through the blood vessel 100 until the portion of the stent delivery system containing the stent 20 is located at the treatment site 102, as shown in FIG. 5B. The balloon 50 is then inflated into contact with the wall 104 of the body vessel 100, which inhibits or prevents blood flow within the blood vessel 100 at the treatment site 102.

Referring to FIG. 5C, after inflating the balloon 50 to inhibit or prevent blood flow at the treatment site 102, the outer sheath 60 is retracted, exposing the distal end 21 of the stent 20 and thus allowing the distal end 21 of the stent 20 to expand.

As shown in FIG. 5D, the outer sheath 60 is retracted until it reaches a position proximal to the bumper member 45. At tins point, the proximal end 22 of the stent 20 becomes exposed and thus the stent 20 expands to a fully deployed diameter contacting the vessel wall 104. The balloon 50 remains inflated throughout the deployment process, and thus inhibits or prevents distal, movement of the stent 20 during deployment. As a result, any distal movement of the stent 20 that might have resulted from stent jumping when using certain traditional stent delivery systems can be limited or prevented.

With the stent 20 fully deployed, the outer sheath 60 is advanced into the lumen formed by the expanded stent 20, as shown in FIG. 5E. A suction device (not shown) is connected to the aspiration connector 67 (FIG. 1) such that the suction device is in fluid communication with the aspiration lumen 61. The suction device is activated to draw blood containing loose debris 106 (e.g., plaque particles loosened from the blood vessel wall during deployment of the stent 20) away from the deployment, site 102 through the opening 62 at the distal end 63 of the outer sheath 60 and into aspiration lumen 61. The blood and debris can be drawn into the suction device and then disposed of.

Next, as shown in FIG. 5F, the balloon 50 is deflated to a size smaller than an inner diameter of the expanded endoprosthesis 20. The stent delivery system 10 is then withdrawn from the body vessel.

While certain embodiments have been described above, other embodiments are possible.

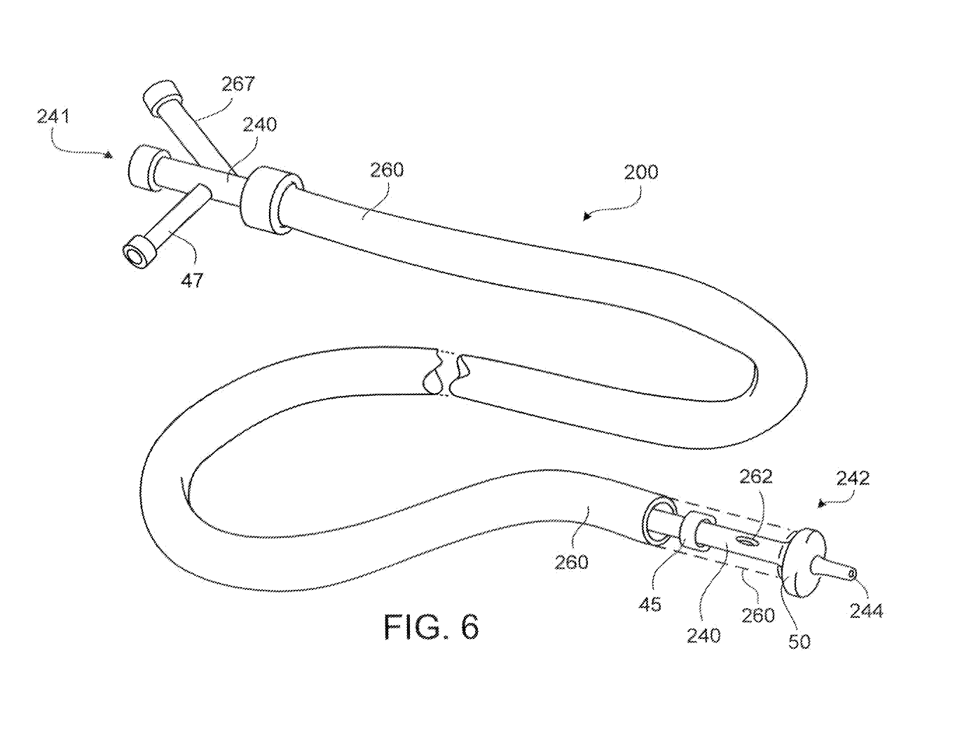

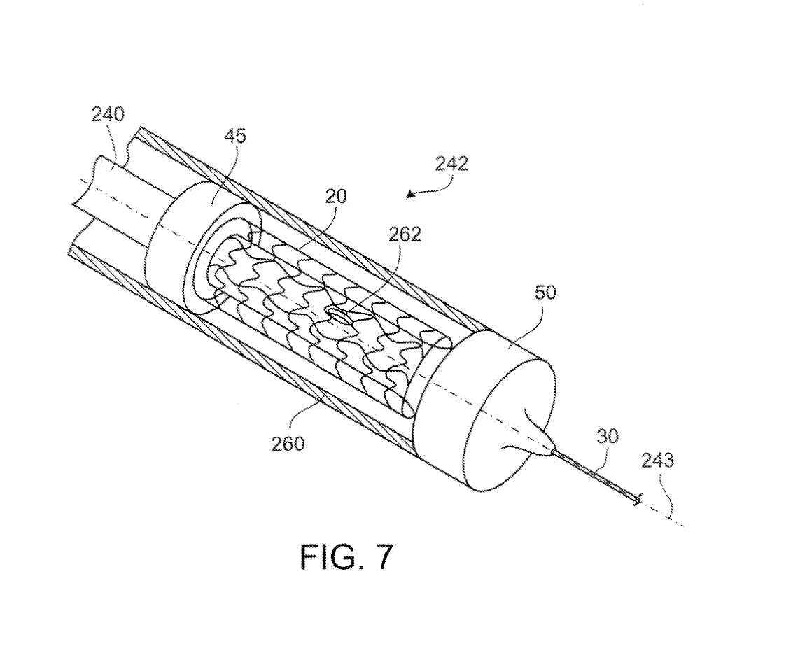

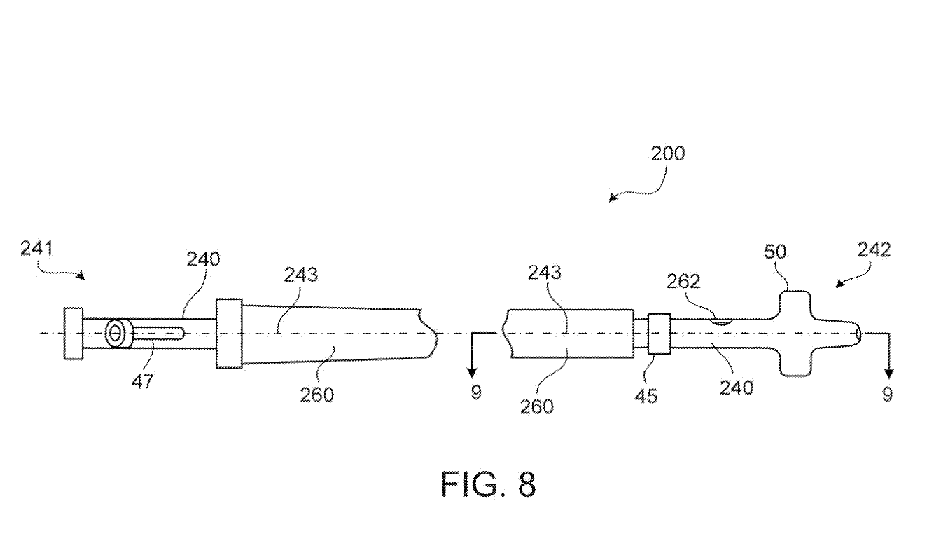

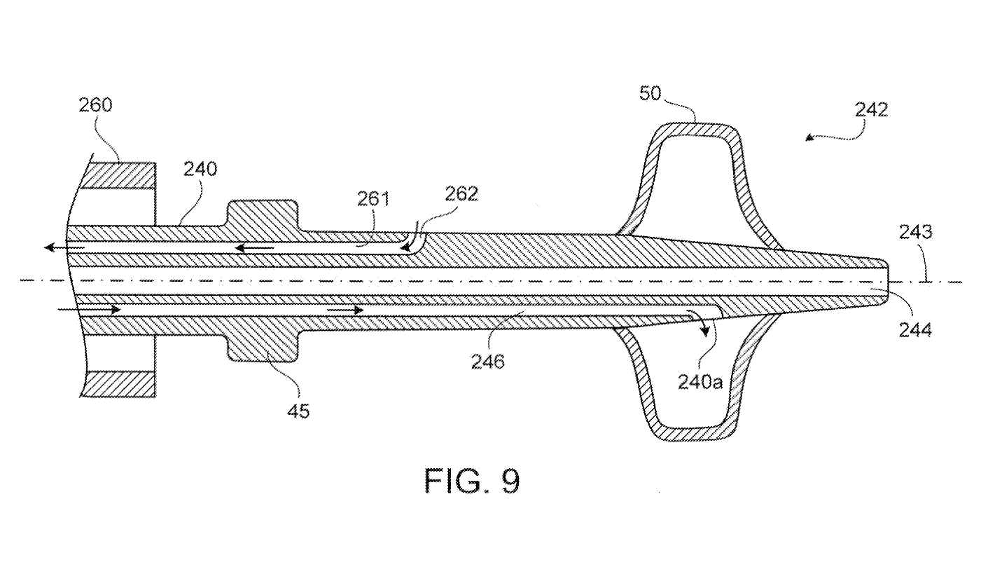

As an example, while the stent delivery systems of the embodiments described above include an aspiration lumen formed by the outer sheath, other arrangements are possible. FIGS. 6-9, for example, illustrate a stent delivery system 200 that includes an inner member 240 that includes an aspiration lumen 261 (FIG. 9). Referring to FIG. 6, the stent delivery system 200 further includes an outer sheath 260 that surrounds the inner member 240. During use, as shown in FIG. 7, the stent 20 can be contained between the outer sheath 260 and the inner member 240.

Referring again to FIG. 6, the inner member 240 has a proximal end 241, a distal end 242 and a longitudinal axis 243 extending therebetween. The balloon 50 and the bumper member 45 are secured to the inner member 240 in a distal region of the inner member 240. The balloon 50 and bumper member 45 are spaced apart along the longitudinal axis 243 and configured to inhibit axial movement of the stent 20 during deployment of the stent 20.

As shown in FIG. 9, the inner member 240 includes the aspiration lumen 261, a guide wire lumen 244, and an inflation lumen 246. The aspiration lumen 261 extends from an opening 262 in the side wall of the inner member 240 toward the proximal end 241 of the inner member 240. The opening 262 is positioned between the bumper member 45 and the balloon 50. The opening 262 and the aspiration lumen 261 can be sized to allow particles dislodged from a blood vessel during deployment of the stent 20 therein to pass through the opening 262 and the aspiration lumen 261. For example, the opening 262 and the aspiration lumen 261 can have a cross-sectional area of about 0.1 mm² to about 4 mm². An aspiration port 267 (FIG. 6) is secured to a proximal region of the inner member 240 and is in fluid communication, with the aspiration lumen 261. A suction device (not shown) can be connected to the aspiration port 267 and activated in order to create a vacuum through the aspiration lumen 261.

During use, a guide wire is positioned in a blood vessel, of a patient, and the stent delivery system 200 is advanced over the guidewire in a manner such that the guidewire extends within the guidewire lumen 244 of the inner member 240. The stent delivery system 200 is positioned at a treatment site within the blood vessel, where the balloon 50 is inflated. The outer sheath 260 is then retracted to deploy the stent 20 within a region of the blood vessel proximal to the inflated balloon 50. After deploying the stent 20 within the blood vessel, the suction device is connected to the aspiration port 267 and activated to draw blood into the aspiration lumen 261 via the opening 262. Because the opening 262 will be positioned within the lumen of the expanded stent 20, any debris (e.g., plaque) dislodged from the wall of the blood, vessel as a result of the deployment of the stent 20 will generally be within suction range of the opening 262 without having to reposition, the inner member 240. As a result, debris that was dislodged from the blood vessel wall during deployment of the stent 20 can be removed from the blood vessel by drawing blood from the treatment site into the suction device via the aspiration lumen 261. Subsequently, the balloon 50 is deflated and the stent delivery system 200 is removed from the blood vessel.

While the stent delivery systems described above include only a single aspiration lumen, in some embodiments, the systems can include multiple aspiration lumens. For example, the systems can include multiple aspiration lumens that extend through and are circumferentially spaced about the inner member. The inner member can also include multiple circumferentially spaced openings that fluidly connect the aspiration lumens to the exterior surroundings of the inner member. The circumferentially spaced relationship between the openings can permit blood and debris to be removed from various different circumferential locations within the blood vessel without having to move (e.g. rotate) the inner member. Alternatively or additionally, the systems can include one or more aspiration lumens formed by the inner member and at least one additional aspiration lumen formed by the outer sheath.

In certain embodiments, the inner member of the stent delivery system includes a single aspiration lumen and multiple, circumferentially spaced openings that fluidly connect the aspiration lumen to the exterior surroundings of the inner member. The circumferentially spaced relationship between the openings can permit blood and debris to be removed from various different circumferential locations within the blood vessel without having to move (e.g., rotate) the inner member.

While the stent delivery systems described above include a balloon sized to prevent blood from flowing through the blood vessel during deployment of the stent, the balloon need not be sized to prevent blood flow. For example, in some embodiments the balloon is sized only to inhibit and/or prevent axial movement of the stent during deployment without substantially inhibiting the flow, of blood through the vessel.

While certain embodiments above describe partially inflating the balloon and abutting the distal end of the outer sheath against the proximal end of the balloon while advancing the stent delivery system through a blood vessel in order to improve trackability of the system, other arrangements can be used to enhance the trackability of the system. In some embodiments, for example, in addition to the balloon, the inner member includes a tip attached to its distal end region. The tip can be arranged to abut the distal end of the outer sheath during delivery to improve trackability of the system. The tip can, for example, have an outer diameter that is greater than, or equal to the outer diameter of the outer sheath. In such, embodiments, the balloon, during delivery, can be deflated and disposed within the outer sheath.

While the stent delivery systems described above include a balloon sized to prevent fluid flow through a blood vessel during deployment of the endoprosthesis and/or to prevent distal movement of the stent during deployment of the endoprosthesis, other types of expandable member can be used to achieve these results. For example, in some embodiments, the system can include an expandable, substantially fluid-Impermeable sheath. The expandable sheath can, for example, include an expandable Nitinol frame surrounded by a fluid-impermeable covering. The frame can include multiple struts affixed to the inner member. The struts can be configured to expand from a relatively small constrained diameter to a relatively large expanded diameter. The fluid-impermeable covering can be attached to the struts, e.g., with, adhesives, solvent bonding, thermal bonding, or combinations thereof. In some embodiments, the expandable sheath is configured to substantially prevent fluid flow through the body vessel when it is expanded in the body vessel. For example, the expandable sheath can be configured to expand to a diameter that is almost 75 percent to about 120 percent of the inner diameter of the body vessel in which it is used. In some embodiments, the struts can extend (e.g., taper) outwardly from the inner member to define a proximal surface, distal to the distal end of the expandable endoprosthesis, to inhibit and/or prevent distal movement of the endoprosthesis during deployment. While the expandable sheath has been described as including a fluid-impermeable covering, the covering can alternatively be formed of a fluid-permeable material, such as mesh.

While the inner members of the stent delivery systems described above are single tubular members, in some embodiments, the inner member includes two co-axial tubes, i.e., inner and outer tubes, in such embodiments, the proximal end region of the balloon is attached to the outer tube and the distal end region of balloon is attached to the inner tube, and an annular inflation lumen is defined between the inner and outer tubes.

While the lumens 61 and 261 of the stent delivery systems described above have been described as being used to aspirate fluid from the treatment site, these lumens can alternatively or additionally be used for other purposes. In some embodiments, for example, these lumens can be used to deliver one or more therapeutic agents, such as paclitaxel, to the treatment site. For example, an ejection device, such as a syringe, containing the one or more therapeutic agents can be placed in fluid communication with the lumen (e.g., by securing the ejection device to the adaptor 67 or 267 of the system). The ejection device can then be activated to expel the one or more therapeutic agents from the ejection device to the treatment site via the lumen. The therapeutic agent(s) can be delivered to the treatment site prior to deployment of the stent, during deployment of the stent, or after deployment of the stent.

While the therapeutic agent(s) have been described as being delivered through lumens 61 and 261, the lumens used to deliver the therapeutic agent(s) can alternatively be separate from those lumens that are used to provide aspiration. In certain embodiments, the therapeutic agent delivery lumen is formed between the outer sheath and the inner member, e.g., extending from an opening at a distal end of the outer sheath toward a proximal end of the outer sheath. Alternatively or additionally, the inner member can include a therapeutic agent delivery lumen for delivering a therapeutic agent to the treatment site.

As noted above the therapeutic agent delivered to the treatment site can be paclitaxel. However, any of various other therapeutic agents can alternatively or additionally be used. Therapeutic agents include agents that are negatively charged, positively charged, amphoteric, or neutral. Therapeutic agents include genetic therapeutic agents, non-genetic therapeutic agents, and cells, and can be negatively charged, positively charged, amphoteric, or neutral. Therapeutic agents can be, for example, materials that are biologically active to treat physiological or pathological conditions: pharmaceutically active compounds; gene therapies; nucleic acids with and without carrier vectors; oligonucleotides; gene/vector systems; DNA chimeras; compacting agents (e.g. DNA compacting agents); viruses; polymers; hyaluronic acid; proteins (e.g., enzymes such as ribozymes); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Non-limiting examples of therapeutic agents include anti-thrombogenic agents; antioxidants; angiogenic and anti-angiogenic agents and factors; anti-proliferative agents (e.g., agents capable of blocking smooth muscle cell proliferation); calcium entry blockers; and survival genes which protect against cell death. In certain embodiments, the therapeutic agent is an immunosuppressant, such as sirolimus.

While the stent delivery systems described above are adapted to deliver self-expanding stents, the systems can be used to deliver other types of implantable medical endoprosthesis. In some embodiments, for example, the systems are configured to deliver balloon expandable stents. In such embodiments, in addition to a balloon that is inflatable to prevent distal movement of the stent and/or to prevent blood flow through the vessel during deployment of the stent, the inner member can include a balloon positioned on the stent bed region of the inner member. A balloon expandable stent can be mounted, e.g., crimped, about the balloon on the stent bed region to permit expansion of the balloon expandable stent during use. Thus, during use, the more distal balloon would first be inflated to prevent distal movement of the stent and/or to prevent blood flow through the vessel, and then the more proximal balloon would be inflated to deploy the stent.

As an alternative to or in addition to self-expanding stents and balloon expandable stems, other types of implantable endoprostheses can be delivered using the delivery-systems described herein. Examples of other types of implantable endoprostheses include grafts, stent-grafts, annuloplasty rings, urolume endoprostheses, etc.

While methods described above include delivering and deploying implantable endoprostheses within blood vessels, the endoprostheses can be delivered and deployed in other types of body lumens. Examples of other types of body lumens include bile ducts, esophagus, colon, trachea or large bronchi, ureters, urethra and/or cardiac valves.

While methods of using the stent delivery systems described above include the use of a guide wire, in some embodiments, the stent delivery systems can be used without a guide wire. In certain embodiments, for example, the inner member of the stent delivery system does not include a guide wire lumen, thereby freeing up additional space in the inner member for larger and/or additional inflation lumens and/or aspiration lumens.

Other embodiments are in the claims.

What is claimed is:

1. A system, comprising:
an inner member;
an outer sheath at least partially surrounding the inner member, the inner member and the outer sheath being configured to receive an implantable endoprosthesis therebetween;
an expandable member secured to the inner member, the expandable member being configured to inhibit fluid flow through a portion of a body vessel when the expandable member is expanded within the body vessel; and
a lumen in fluid communication with a region of the body vessel adjacent the expandable member during use, wherein fluid can be withdrawn from the body vessel through the lumen during use.

2. His system according to claim 1, wherein the lumen is defined by the outer sheath.

3. The system according to claim 1, wherein the lumen is defined by the inner member.

4. The system according to claim 1, wherein the lumen can be placed in fluid communication with the region of the body vessel adjacent the expandable member by proximally displacing the outer sheath, relative to the inner member.

5. The system according to claim 1, wherein the lumen is configured to allow particles dislodged from a wall of the body vessel while deploying an implantable endoprosthesis within the body vessel to pass through the lumen.

6. The system according to claim 1, further comprising a suction device in communication with the lumen, the suction device being capable of drawing fluid from the region of the body vessel adjacent the expandable member via the lumen.

7. The system according to claim 1, wherein the expandable member is configured to substantially prevent fluid flow through the body vessel when expanded therein.

8. The system according to claim 1, wherein the expandable member is configured to substantially prevent distal movement of an implantable endoprosthesis disposed between the inner member and the outer sheath during deployment of the implantable endoprosthesis.

9. The system according to claim 8, wherein the implantable endoprosthesis can be deployed by proximally displacing the outer sheath relative to the inner member.

10. The system according to claim 1, wherein the implantable endoprosthesis comprises a self-expanding stent.

11. The system according to claim 1, wherein the expandable member comprises a balloon.

12. A system, comprising:
an inner tubular member;
an outer sheath at least partially surrounding the inner tubular member;
a self expanding implantable endoprosthesis disposed between the inner tubular member and the outer sheath;
an expandable member secured to the inner tubular member, a proximal face of the expandable member being disposed distal to a distal end of the self-expanding implantable endoprosthesis, the expandable member being configured, when expanded, to substantially prevent distal movement of the self-expanding implantable endoprosthesis during deployment of the self-expanding implantable endoprosthesis.

13. The system according to claim 12, wherein the expandable member, when expanded, has a diameter that is at least about 75 percent of a diameter of the self-expanding implantable endoprosthesis when fully deployed.

14. The system according to claim 12, wherein the proximal race of the expandable member extends at an angle of about 45 degrees to about 90 degrees relative to a longitudinal, axis of the inner member when the expandable member is expanded.

15. The system according to claim 12, wherein the proximal face of the expandable member is positioned within about 5 millimeters of the distal end of the self-expanding implantable endoprosthesis.

16. The system according to claim 12, wherein, the expandable member is configured to inhibit fluid flow through a region of a body vessel when expanded therein.

17. The system according to claim 16, further comprising a suction device in communication with a lumen, the suction device being capable of drawing fluid from a region of the body vessel proximal to the expandable member via the lumen.

18. The system according to claim 17, wherein the lumen is defined by the outer sheath.

19. The system according to claim 17, wherein, the lumen is defined by the inner member.

20. The system according to claim 12, wherein the self-expanding implantable endoprosthesis comprises a self-expanding stent.

21. The system according to claim 12, wherein the expandable member comprises a balloon.

22. A method, comprising:
expanding an expandable member within a body vessel, the expanded expandable member inhibiting fluid flow through a portion of the body vessel;
after expanding the expandable member, deploying an implantable endoprosthesis within the body vessel adjacent the expanded expandable member; and
withdrawing fluid from a region of the body vessel adjacent the expanded expandable member.

23. The method according to claim 22, wherein the expanded expandable member substantially prevents fluid flow through the body vessel.

24. The method according to claim 22, wherein the lumen is defined by the outer sheath.

25. The method according to claim 22, wherein the lumen is defined by the inner member.

26. The method according to claim 22, wherein the fluid is withdrawn from the body vessel after deploying the implantable endoprosthesis.

27. The method according to claim 26, wherein the fluid comprises particles dislodged from the body vessel during deployment of the implantable endoprosthesis.

28. The method according to claim 22, wherein deploying the implantable endoprosthesis comprises retracting an outer sheath relative to an inner member, the implantable endoprosthesis, prior to deployment, being disposed between the inner member and the outer sheath.

29. The method according to claim 28, wherein a proximal face of the expandable member is positioned distal to a distal end of the implantable endoprosthesis.

30. The method according to claim 22, wherein the expanded expandable member is configured to substantially prevent distal movement of the implantable endoprosthesis during deployment of the endoprosthesis.

31. The method of claim 22, further comprising delivering a therapeutic agent through the lumen to the region of the body vessel adjacent the expanded expandable member.
